(19) United States
(12) Patent Application Publication
(10) Pub. No.: US 2023/0407260 A1
(43) Pub. Date: Dec. 21, 2023

(54) PARTICULAR CARDIAC TISSUE AND ITS USE IN THE TREATMENT OF CARDIAC PATHOLOGIES

(71) Applicant: TREEFROG THERAPEUTICS, PESSAC (FR)

(72) Inventors: Maxime FEYEUX, TALENCE (FR); Andrea LEONARD, PESSAC (FR)

(21) Appl. No.: 18/027,209

(22) PCT Filed: Sep. 21, 2021
(86) PCT No.: PCT/EP2021/075951

§ 371 Date: Jun. 14, 2023

(30) Foreign Application Priority Data
Sep. 21, 2020 (FR) FR2009553

(51) Int. Cl. C12N 5/077; C12N 5/00; A61P 9/10

(52) U.S. Cl. CPC C12N 5/0657; C12N 5/0012; C12N 2527/00; C12N 2513/00; A61P 9/10

(57) ABSTRACT

The invention relates to a compacted tissue of human cardiac cells expressing cardiac troponin C, which is contractile and has a low spontaneous contraction frequency. The invention also relates to the use of the tissue, in particular in the treatment of a cardiac pathology, particularly ischaemic heart disease.

### TECHNICAL FIELD

The invention relates to the treatment of heart diseases, in particular ischemic heart diseases, by the use of specific cardiac tissues obtained from specific cellular microcompartments comprising human cells expressing genes which are expressed during cardiac differentiation

### PRIOR ART

According to the World Health Organization, cardiovascular diseases, and in particular ischemic heart diseases (which generally lead to myocardial infarction), are the main cause of death in the world (Thomas, H. et al. Global Atlas of Cardiovascular Disease 2000-2016: The Path to Prevention and Control. Glob. Heart 13, 143-163 (2018)).

Currently, there is no satisfactory solution for preventing or treating the consequences of cardiac ischemia and in particular for treating necroses of the cardiac muscle, which are responsible for heart failure and for risks of cardiac arrest.

Recently, research has been carried out into the use of cardiomyocytes derived from human pluripotent stem cells, hPSC-CMs, which comprise both human embryonic stem cells and induced pluripotent stem cells, to regenerate lost or damaged cardiac tissues in order to prevent or treat the associated heart failure (Desgres, M. & Menasche, P. Clinical Translation of Pluripotent

Stem Cell Therapies: Challenges and Considerations. Cell Stem Cell 25, 594-606 (2019); Bertero, A. & Murry, C. E. Hallmarks of cardiac regeneration. Nat. Rev. Cardiol. 15, 579-580 (2018); Jiang, B., Yan, L., Shamul, J. G., Hakun, M. & He, X. Stem Cell Therapy of Myocardial Infarction: A Promising Opportunity in Bioengineering. Adv. Ther. 3, 1900182 (2020); Liew, L. C., Ho, B. X. & Soh, B. S. Mending a broken heart: Current strategies and limitations of cell-based therapy. Stem Cell Res. Ther. 11, 1-15 (2020)).

These cardiomyocytes can be used for other applications, particularly as biological cardiac stimulators for treating sinus node dysfunction (Lee, J. H., Protze, S. I., Laksman, Z., Backx, P. H. & Keller, G. M. Human Pluripotent Stem Cell-Derived Atrial and Ventricular Cardiomyocytes Develop from Distinct Mesoderm Populations. Cell Stem Cell 21, 179-194.e4 (2017)), or for treating congenital heart diseases such as septal defects (Devalla, H. D. & Passier, R. Cardiac differentiation of pluripotent stem cells and implications for modeling the heart in health and disease. Sci. Transl. Med. 10, 1-14 (2018)) or else for disease modeling, to test candidate medicaments (Tzatzalos, E., Abilez, O. J., Shukla, P. & Wu, J. C. Engineered heart tissues and induced pluripotent stem cells: Macro- and microstructures for disease modeling, drug screening, and translational studies. Adv. Drug Deliv. Rev. 96, 234-244 (2016)).

It is estimated that the amount of cells needed to regenerate damaged cardiac tissues of a patient after myocardial infarction is approximately 1 billion (Laflamme, M. A. & Murry, C. E. Heart regeneration. Nature 473, 326-335 (2011)), meaning it is currently impossible to use hPSC-CM-derived cardiomyocytes in cardiac cell therapy. Indeed, producing cardiac tissues on an industrial scale is complex because it is necessary to achieve a compromise between culture conditions that are sufficiently mild for the survival and correct functioning of the tissues and large-volume culture constraints that inevitably expose the cells to non-physiological stresses (typically hydrodynamic stress in the context of liquid culturing in bioreactors). The methods for producing cardiomyocytes from hPSC have the following problems, in particular:
Poor formation of hPSC aggregates in cultures in suspension prior to differentiation into cardiomyocytes: indeed, the initial formation of the hPSC aggregates, and homogeneity, are crucial for cell reproduction and therefore for the quality of the cardiac tissue obtained after differentiation;
A significant loss of cells due to the sensitivity of the hPSCs to shear stress and to impacts during culturing in a bioreactor (Lam, A. T. L. et al. Conjoint propagation and differentiation of human embryonic stem cells to cardiomyocytes in a defined microcarrier spinner culture. Stem Cell Res. Ther. 1-15 (2014));
The impossibility of combining large-scale amplification of the hPSCs and cardiac differentiation (Le, M. N. T. & Hasegawa, K. Expansion culture of human pluripotent stem cells and production of cardiomyocytes. Bioengineering 6, (2019)).

Known solutions for limiting these disadvantages during cardiac differentiation in suspension in a bioreactor require:
either using microcarriers and temporarily stopping agitation (Ting, S., Chen, A., Reuveny, S. & Oh, S. An intermittent rocking platform for integrated expansion and differentiation of human pluripotent stem cells to cardiomyocytes in suspended microcarrier cultures. Stem Cell Res. 13, 202-213 (2014))
or using cell lines that are less sensitive to shearing during cardiac differentiation (Laco, F. et al. Selection of human induced pluripotent stem cells lines optimization of cardiomyocytes differentiation in an integrated suspension microcarrier bioreactor. Stem Cell Res. Ther. 11, 1-16 (2020)).

However, these solutions are not optimal. In particular:
the micro-carriers still leave the cells exposed to mechanical stresses which may be difficult to remove,
stopping agitation does not allow for uniform diffusion of the nutrients and products necessary for differentiation, and
limiting to a single starting cell line is extremely restrictive and limiting.

It is also known that the loss of cells undergoing differentiation into cardiomyocytes can also be reduced by performing culture directly in bulk hydrogel (Kerscher, P. et al. Direct Production of Human Cardiac Tissues by Pluripotent Stem Cell Encapsulation in Gelatin Methacryloyl. ACS Biomater. Sci. Eng. 3, 1499-1509 (2017); Li, Q. et al. Scalable and physiologically relevant microenvironments for human pluripotent stem cell expansion and differentiation. Biofabrication 10, (2018)), but these methods are not compatible with conventional bioreactor culturing. In order to make the method compatible with bioreactors used in industry, tests were carried out by encapsulating the cells in the hydrogel, but these tests were limited to mouse stem cells (Agarwal, P. et al. A Biomimetic Core-Shell Platform for Miniaturized 3D Cell and Tissue Engineering. Part. Part. Syst. Charact. 32, 809-816 (2015), Chang, S. et al. Emulsion-based Encapsulation of Pluripotent Stem Cells in Hydrogel Microspheres for Cardiac Differentiation. Biotechnol. Prog. btpr.2986 (2020) doi:10.1002/btpr.2986; Zhao, S. et al. Bioengineering of injectable encapsulated aggregates of pluripotent stem cells for therapy of myocardial infarction. Nat. Commun. 7, 1-12 (2016)).

Finally, it is known that cell retention of the cardiomyocytes after delivery into the heart is very poor (Hou, D. et al. Radiolabeled cell distribution after intramyocardial, intracoronary, and interstitial retrograde coronary venous delivery: Implications for current clinical trials. Circulation 112, 150-156 (2005)), which limits the efficacy of cardiac cell therapy. In addition, during the grafting of cardiomyocytes as are obtained currently, in order to regenerate cardiac tissues, there is a significant risk of inducing an arrhythmia in the patient, which again limits the use of the cell therapy for treating cardiac pathologies. Indeed, to date, one of the most crucial scientific challenges to be overcome in order to ensure the safety of stem cell-derived cardiac cells for therapeutic applications related to heart diseases is the elimination of the arrhythmia induced during the graft (Menasché, P. Cardiac cell therapy: Current status, challenges and perspectives. Archives of Cardiovascular Diseases 113, 285-292 (2020); Kadota, S., Tanaka, Y. & Shiba, Y. Heart regeneration using pluripotent stem cells. Journal of Cardiology (2020) doi:10.1016/j.jjcc.2020.03.013; Chen, K., Huang, Y, Singh, R. & Wang, Z. Z. Arrhythmogenic risks of stem cell replacement therapy for cardiovascular diseases. Journal of Cellular Physiology (2020) doi:10.1002/jcp.29554). Although generally transient, arrhythmias were observed both in pigs (Romagnuolo, R. et al. Human Embryonic Stem Cell-Derived Cardiomyocytes Regenerate the Infarcted Pig Heart but Induce Ventricular Tachyarrhythmias. Stem Cell Reports 12, 967-981 (2019)) and in non-human primates (Ichimura, H. et al. Allogeneic transplantation of iPS cell-derived cardiomyocytes regenerates primate hearts. Nature 538, 388-391 (2016); Liu, Y.-W. et al. Human embryonic stem cell-derived cardiomyocytes restore function in infarcted hearts of non-human primates. Nature biotechnology 36, 597-605 (2018); Chong, J. J. H. et al. Human embryonic-stem-cell-derived cardiomyocytes regenerate non-human primate hearts. Nature 510, 273-277 (2014)), which are important animal models for regeneration following myocardial infarction.

There is therefore a great need for a solution that enables the large-scale production of quality cardiomyocytes, in order to meet a crucial need for cardiac cell therapy, but also in the research and development of medicament molecules in order to assess their efficacy and their toxicity in the preclinical phase, before exposing patients to these treatments.

The aim of the invention is therefore to meet all of these needs and to overcome the disadvantages and limits of the prior art.

### SUMMARY OF THE INVENTION

In order to achieve this aim, the inventors developed a specific cardiac tissue.

The specific subject of the invention is a compacted tissue of human cardiac cells expressing cardiac troponin C (that is to say, human cells expressing the cardiac troponin C gene, the alias of the corresponding gene being TNNC1), which is contractile and has a spontaneous contraction frequency of less than 4 Hz.

Another subject of the invention is a compacted tissue of human cardiac cells expressing cardiac troponin C (that is to say, human cells expressing the cardiac troponin C gene, the alias of the corresponding gene being TNNC1).

Preferentially, the compacted tissue of human cardiac cells according to the invention has a content of cells expressing cardiac troponin C of at least 50% by number relative to the total number of cells forming the compacted tissue, even more preferentially at least 60%, at least 70%, at least 75%, at least 80%, it being possible for this content to be greater than 90%.

Preferentially, the compacted tissue of human cardiac cells according to the invention has a content of cells expressing alpha-actinin of at least 50% by number relative to the total number of cells forming the compacted tissue, even more preferentially at least 60%, at least 70%, at least 75%, at least 80%, it being possible for this content to be greater than 90%. Preferentially, the compacted tissue of human cardiac cells according to the invention has a content of cells expressing troponin C and alpha-actinin of at least 50% by number relative to the total number of cells forming the compacted tissue, even more preferentially at least 60%, at least 70%, at least 75%, at least 80%, it being possible for this content to be greater than 90%.

In the prior art, Jing Donghui et al. “Cardiac cell generation from encapsulated embryonic stem cells in static and scalable culture systems”, Cell Transplantation, col. 19, no 11, 1 Nov. 2010, pages 1397-1492 described cardiac tissues obtained by encapsulating mouse or human ESC in alginate beads coated with polylysine, then dissolution of the core by incubation in a sodium citrate solution. This solution, demonstrated using embryonic stem cells, is unsatisfactory: the purity of the tissue obtained, and in particular the content of cells expressing cardiac troponin C, is less than 20%, which is insufficient to be considered for therapeutic use. With too many cellular impurities in tissues, said tissues present a risk for the patient by introducing undesired cells into the cardiac muscle, with the risk of disrupting the correct functioning thereof, in particular by disrupting the electrical conductivity and/or the contractability thereof. In addition, the use of polylysine at the concentrations described in this article presents a risk of toxicity for the cells.

Likewise, Koivisto Janne T. et al. “Mechanically Biomimetic Gelatin-Gellan Gum Hydrogels for 3D Culture of Beating Human Cardiocytes”, Applied Materials & Interfaces, vol. 11, no. 23, 12 Jun. 2019, pages 20589-20602 describes the encapsulation of cells within a hydrogel without space available for the organization of the cells, even more so after cell proliferation which mechanically pressurizes the hydrogel. There is no encapsulation in microcompartments and this does not lead to a suitable contraction frequency, nor to a content of cells expressing cardiac troponin C that is sufficient for therapeutic use.

The cardiac tissues according to the invention, obtained by a specific method different from those of the prior art, makes it possible to obtain cardiac tissues with a content of cells expressing cardiac troponin C and/or alpha-actinin of at least 50%, and preferentially of at least 75%. Indeed, the configuration according to the invention while undergoing differentiation enables the transmission of autocrine/paracrine signals within a protected lumen, which enables the cells to self-organize in a manner which is biomimetic of the in vivo structure. This structure is extremely fragile and requires both mechanical protection and available space, contrary to that described in Koivisto Janne T et al. According to the invention, this configuration cannot be established either in a confined system or in an unprotected system. Indeed, cardiac differentiation is notoriously difficult to reproduce in vitro in conventional systems (both 3D and 2D, as demonstrated in particular in https://www.sciencedirect.com/science/article/pii/S2213671118301504) which reflects incomplete control of the cell environment. In a non-obvious and surprising manner, the invention proposes controlled structuring of the environment in the form of protected self-organization, enabling lower sensitivity to small variations in the culture system and therefore greater reproducibility.

Advantageously, these cardiac tissues, used as is or after dissociation into cells, are suitable for uses in cell therapy. In particular, these tissues can be used to regenerate ischemic cardiac tissues.

Thus, the invention targets said tissues, as is or after dissociation into cells, for use thereof in the prevention and/or treatment of pathologies, in particular cardiac pathologies.

In order to obtain this compacted tissue of human cardiac cells, according to one embodiment, it is possible to proceed via a key developmental intermediate, namely a three-dimensional (3D) cellular microcompartment comprising in succession, organized around at least one lumen:
at least one inner layer of human cells undergoing cell differentiation into cardiac cells, expressing at least one gene selected from PDGFRα, MESP-1, NKX2-5, GATA4, MEF2C, TBX20, ISL1 and TBX5, said inner layer having a variable thickness;
at least one intermediate layer of isotonic aqueous solution, and
at least one external hydrogel layer.

A microcompartment of this kind thus comprises cells undergoing cell differentiation, with the expression of the genes PDGFRa/MESP1/NKX2-5/GATA4/MEF2C/TBX20/ISL1/TBX5 being associated with intermediate stages in cardiac differentiation. This configuration (one or more lumens around which are organized, in succession, a layer of human cells undergoing cardiac differentiation with specific thickness characteristics, a layer of isotonic aqueous solution, and at least one hydrogel layer) is novel. Indeed, there are several known protocols for differentiating hPSCs into cardiomyocytes, which protocols are based partially or entirely on modulating the Wnt (Wingless and Int-1) pathway (Dunn, K. K. & Palecek, S. P. Engineering scalable manufacturing of high-quality stem cell-derived cardiomyocytes for cardiac tissue repair. Front. Med. 5, (2018)). During the directed cardiac differentiation of hPSCs, the cells undergo morphological changes during their transition to mesoderm, cardiac mesoderm, cardiac progenitor cells and finally to cardiac myocytes. In 2D hPSC culture, these changes are known to be associated with distinct morphologies (Palpant, N. J. et al. Generating high-purity cardiac and endothelial derivatives from patterned mesoderm using human pluripotent stem cells. Nat. Protoc. 12, 15-31 (2017)). These morphological changes have not been well described in a 3D culture system, and the topology which is a subject of the invention has never been obtained and described. This advantageously then makes it possible to obtain large amounts of compacted tissues having characteristics that enable the use thereof to regenerate damaged cardiac tissues.

The presence of an external hydrogel layer and an intermediate layer of isotonic aqueous solution enables uniform distribution of the cells between the microcompartments. Thus, the homogeneity between the microcompartments is greatly improved by the prior encapsulation of the hPSCs, enabling increased yield and quality compared with existing methods. Moreover, this hydrogel layer makes it possible to prevent microcompartments from fusing, these fusion events being a major source of variability which is unfavorable for phenotypic homogeneity and for the survival of cardiac cells produced in a bioreactor.

In addition, modulation of the Wnt pathway used in cardiac differentiation is associated with degradation of β-catenin (Lam, A. T. L. et al. Conjoint propagation and differentiation of human embryonic stem cells to cardiomyocytes in a defined microcarrier spinner culture. Stem Cell Res. Ther. 1-15 (2014)), a molecule that plays a role in cell-cell adhesion complexes (Brembeck, F. H., Rosario, M. & Birchmeier, W. Balancing cell adhesion and Wnt signaling, the key role of β-catenin. Curr. Opin. Genet. Dev. 16, 51-59 (2006)). Advantageously, the topology of the microcompartment makes it possible to protect the cells undergoing cardiac differentiation, despite the fragility of the cell-cell adhesion induced by the modulation of the Wnt pathway.

Other features and advantages will emerge from the detailed description of the invention and the following examples.

### BRIEF DESCRIPTION OF THE FIGURES

FIG. 1a is a schematic depiction of a sectional view of compacted cardiac tissue 22 according to the invention, corresponding to the photo shown in FIG. 1b, with an external hydrogel layer 12, a layer of isotonic aqueous solution 14, a compacted tissue of differentiated cardiac cells 20.

FIG. 1b is a phase contrast microscopy image of a compacted tissue according to the invention in a microcompartment, taken at 4× magnification which corresponds to the schematic diagram of FIG. 1a.

FIG. 1c shows phase contrast microscopy images taken at 4× magnification of compacted tissues according to the invention in microcompartments.

FIG. 2a is a schematic depiction of a sectional view of a cellular microcompartment 10, corresponding to the photo shown in FIG. 2b, with an external hydrogel layer 12, a layer of isotonic aqueous solution 14, a layer of human cells undergoing cardiac differentiation 16 with a greatest thickness t2 and a smallest thickness t1, and an inner lumen 18.

FIG. 2b is a phase contrast microscopy image of a microcompartment taken at 4× magnification which corresponds to the schematic diagram of FIG. 2a.

FIG. 3a is a schematic depiction of a sectional view of a cellular microcompartment 10, corresponding to the photo shown in FIG. 3b, with an external hydrogel layer 12, a layer of isotonic aqueous solution 14, a layer of human cells undergoing cardiac differentiation 16 with a greatest thickness t2 and a smallest thickness t1, and two inner lumens 18-1 and 18-2, S1 representing the thickness of the layer of isotonic aqueous solution 14.

FIG. 3b is a phase contrast microscopy image of a microcompartment taken at 4× magnification which corresponds to the schematic diagram of FIG. 3a.

FIG. 3c is a phase contrast microscopy image of a plurality of microcompartments taken at 4× magnification, each microcompartment having different morphologies.

FIG. 4a is a schematic depiction of a sectional view of a cellular microcompartment 10, corresponding to the photograph shown in FIG. 4b, with an external hydrogel layer 12, a layer of isotonic aqueous solution 14, a layer of human cells undergoing cardiac differentiation 16 with a greatest thickness t2 and a smallest thickness t1, and two inner lumens 18-1 and 18-1, s1 representing the thickness of the layer of isotonic aqueous solution 14.

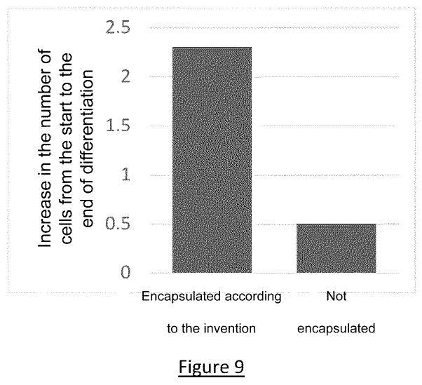

FIG. 4b is a phase contrast microscopy image of a microcompartment taken at 4× magnification which corresponds to the schematic diagram of FIG. 4a. FIG. 5 comprises:
a graph showing the beating frequency of tissues and/or cells obtained from a series of phase contrast microscopy images (at a frequency of at least 30 images per second) on a standard table microscope with 4× magnification, and
phase contrast microscopy images taken at 4× magnification showing (the outermost) encapsulated or free stem cells at the start of differentiation, and the compacted tissues according to the invention approximately 2 weeks after the start of differentiation (A1: Stem cell aggregates without a capsule; A2: Compacted cardiac tissues derived from these aggregates; B1: Compacted cardiac tissue in capsules; B2: microcompartments containing cardiac cells undergoing differentiation; B3: encapsulated stem cells).

FIG. 6 comprises:
a graph showing the beating frequency of tissues and/or cells obtained from a series of phase contrast microscopy images (at a frequency of at least 30 images per second) on a standard table microscope with 4× magnification, and
phase contrast microscopy images taken at 4× magnification. The left-hand image shows the compacted cardiac tissues differentiated in the capsule from encapsulated hiPSCs. The right-hand image shows the cells obtained by dissociating compacted cardiac tissues according to the invention.

FIG. 7 comprises phase contrast microscopy images taken at 4× magnification. The three images on the top row (a, b and c) are images of encapsulated cells. The three images on the bottom row (d, e and f) are images of non-encapsulated cells. The images in the left-hand column (a and d) show stem cells induced at the start of differentiation into cardiac cells. The images in the middle column (b and e) show human cells undergoing cell differentiation into cardiac cells 3 to 7 days after initiation of differentiation. The images in the right-hand column (c and f) show differentiated cardiac tissues.

FIG. 8 is a graph which shows the percentage of cells in the tissues (obtained as in FIGS. 7c and 7f) expressing cardiac troponin C: on the left, encapsulated tissues according to the invention (image 7c); on the right, non-encapsulated tissues (image 7f).

FIG. 9 is a graph which shows the level of cell amplification between the start of differentiation (obtained as in FIGS. 7a and 7d) in the tissues: on the left, encapsulated according to the invention; on the right, non-encapsulated.

### DETAILED DESCRIPTION OF THE INVENTION

### Definitions

For the purposes of the invention, “alginate” means linear polysaccharides formed from β-D-mannuronate and α-L-guluronate, salts and derivatives thereof.

For the purposes of the invention, “hydrogel capsule” means a three-dimensional structure formed from a matrix of polymer chains, swollen using a liquid, preferentially water.

For the purposes of the invention, “cell expressing a gene” means a cell

which contains at least 5 times more copies of the RNA transcribed from the DNA sequence of the gene in question compared to a pluripotent cell, preferentially 10 times more copies, preferentially 20 times more copies, preferentially 100 times more copies.

For the purposes of the invention, “human cells” means human cells or immunologically humanized non-human mammalian cells. Even when this is not specified, the cells, stem cells, progenitor cells and tissues according to the invention consist of or are obtained from human cells or from immunologically humanized non-human mammalian cells.

For the purposes of the invention, “progenitor cell” means a stem cell that is already committed to cell differentiation into cardiac cells but that has not yet differentiated.

For the purposes of the invention, “embryonic stem cell” means a pluripotent stem cell of cells derived from the internal cell mass of the blastocyst. The pluripotency of the embryonic stem cells can be evaluated by the presence of markers such as the transcription factors OCT4, NANOG and SOX2 and surface markers such as SSEA3/4, Tra-1-60 and Tra-1-81. The embryonic stem cells used in the context of the invention are obtained without destroying the embryo from which they originate, for example using the technique described in Chang et al. (Cell Stem Cell, 2008, 2(2)): 113-117). Optionally, embryonic stem cells from humans can be excluded.

For the purposes of the invention, “pluripotent stem cell” or “pluripotent cell” means a cell which has the capacity to form all the tissues present in the entire organism of origin, without however being able to form an entire organism per se. Human pluripotent stem cells can be called hPSC in the present application. These may in particular be induced pluripotent stem cells (iPSC or hiPSC for human induced pluripotent stem cells), embryonic stem cells or MUSE cells (for “multilineage-differentiating stress enduring”).

For the purposes of the invention, “induced pluripotent stem cell” means a pluripotent stem cell induced to become pluripotent by genetic reprogramming of differentiated somatic cells. These cells are in particular positive for pluripotency markers, such as staining with alkaline phosphatase and expression of the proteins NANOG, SOX2, OCT4 and SSEA3/4. Examples of methods for obtaining induced pluripotent stem cells are described in the articles by Yu et al. (Science 2007, 318 (5858): 1917-1920), Takahashi et al (Cell, 207, 131(5): 861-872) and Nakagawa et al (Nat Biotechnol, 2008, 26(1): 101-106).

For the purposes of the invention, “differentiated cardiac cells” means cells which have the phenotype of a cardiomyocyte, that is to say, which express specific markers such as TNNC1 (cardiac troponin C gene) and ACTN2 (alpha-actinin gene) and are capable of spontaneously contracting in response to an intracellular calcium signal in a spontaneous manner (in the case of immature cardiac cells) or following electrical or chemical stimulation capable of triggering said calcium signal.

For the purposes of the invention, “differentiated” cells means cells which have a particular phenotype, as opposed to pluripotent stem cells which are not differentiated.

The “Feret diameter” of a compacted cardiac tissue according to the invention or of a microcompartment means the distance “d” between two tangents to said compacted tissue or to said microcompartment, these two tangents being parallel, such that the whole of the projection of said compacted tissue or of said microcompartment is contained between these two parallel tangents.

For the purposes of the invention, “variable thickness” of the inner layer of human cells undergoing cell differentiation means that, in the same microcompartment, the inner layer does not have the same thickness throughout.

For the purposes of the invention, “implantation” or “graft” in the heart means the action of depositing, at a particular location in the heart, at least one compacted tissue according to the invention. The implantation can be carried out by any means, in particular by injection.

For the purposes of the invention, “microcompartment” or “capsule” means a partially or entirely closed three-dimensional structure containing at least one cell.

For the purposes of the invention, “convective culture medium” means a culture medium stirred by internal movements.

For the purposes of the invention, “largest dimension” of a compacted cardiac tissue according to the invention or of a microcompartment means the value of the largest Feret diameter of said compacted tissue or of said microcompartment.

For the purposes of the invention, “smallest dimension” of a compacted cardiac tissue according to the invention or of a microcompartment means the value of the smallest Feret diameter of said compacted tissue or of said microcompartment.

For the purposes of the invention, “tissue” or “biological tissue” has the common meaning for tissue in biology, that is to say, the intermediate organization level between cell and organ. A tissue is a set of similar cells of the same origin (commonly derived from a common cell line, although they can originate in the association of distinct cell lines), grouped into a cluster, network or bundle (fiber). A tissue forms a functional assembly, that is to say that its cells contribute to the same function. Biological tissues regenerate regularly and are assembled together to form organs.

For the purposes of the invention, “compacted tissue” or “compacted cardiac tissue” or “compacted tissue of cardiac cells” means a tissue unit comprising at least one cardiac tissue consisting at least of differentiated cardiac cells. The tissue is at least partially compacted, that is to say it is composed mainly of cells, in particular its volume is composed of more than 50% of cells, preferentially 75% of cells, preferentially 90% of cells. The tissue can be fully compacted, that is to say the lumens are no longer detectable and/or there are no lumens. The compacted tissues according to the invention can be referred to as microtissues.

For the purposes of the invention, “lumen” means a volume of aqueous solution topologically surrounded by cells. Preferentially, its content is not in diffusive equilibrium with the volume of convective liquid present outside the microcompartment.

### Three-Dimensional Compacted Cardiac Tissue

A subject of the invention is therefore a compacted tissue of human cardiac cells expressing cardiac troponin C (that is to say human cells expressing the cardiac troponin C gene, the alias of the corresponding gene being TNNC1), and preferentially also alpha-actinin.

The tissue according to the invention is therefore a human tissue comprising at least differentiated cardiac cells expressing cardiac troponin C and preferentially alpha-actinin. The compacted tissue according to the invention can also contain other cell types.

The tissue has preferentially been obtained from a tissue of cardiac cells and/or cells undergoing cardiac differentiation comprising at least one lumen, by a method comprising the compaction (“secondary” compaction) of said tissue by the total or partial elimination of said lumens.

This compacted tissue is contractile and has a spontaneous contraction frequency of less than 4 Hz, preferentially less than 2 Hz, even more preferentially less than 1.7 Hz, in particular less than 1 Hz, and particularly less than 0.5 Hz. Advantageously, it may be less than 0.25 Hz. This contraction frequency of the tissue is low, which is a significant advantage for implantation thereof into the heart. Indeed, such a frequency makes it possible to prevent arrhythmia when the compacted tissue according to the invention is grafted into the heart to be treated.

The average heart rate of a human adult is between 60 and 100 beats per minute (1 to 1.7 Hz). The low contraction frequency of the compacted tissues cardiac according to the invention reduces the risk of arrhythmia during grafting of the tissues or cells obtained from these tissues. According to one embodiment, with a spontaneous beating frequency of the tissue according to the invention that is lower than the patient's (recipient's) heart rate, this risk of arrhythmia is further reduced.

The reduction in the spontaneous beating frequency is associated with the maturity of the cardiomyocytes derived from human stem cells, with the 3D culture environment improving the maturation of cardiomyocytes. According to the invention, the encapsulation also makes it possible to reduce the contraction frequency of the compacted cardiac tissue. FIG. 5 shows that, for a given starting cell population, cardiomyocytes differentiated within a microcompartment/capsule (from encapsulated human pluripotent stem cells) have a spontaneous beating rate that is slower than cardiomyocytes differentiated using the same protocol (and the same initial batch of human pluripotent stem cells) but in free suspended culture. Thus, differentiation into cardiomyocytes from encapsulated stem cells reduces the spontaneous contraction frequency.

Human pluripotent stem cells secrete signaling molecules during the cardiac differentiation process, which generate a specific paracrine microenvironment necessary for successful differentiation (Kempf, H. et al. Bulk cell density and Wnt/TGFbeta signalling regulate mesendodermal patterning of human pluripotent stem cells. Nature Communications 7, (2016)). The presence of the capsule helps to increase and to maintain a local concentration of these paracrine factors, which improves the differentiation phenotype, resulting in the reduction of the spontaneous beating frequency.

This contraction frequency of the tissue is low, which is a significant advantage for implantation thereof into the heart. Indeed, such a frequency makes it possible to prevent arrhythmia when the compacted tissue according to the invention is grafted into the heart to be treated.

The compacted cardiac tissue according to the invention can remain spontaneously contractile for several months. Thus, the product is stable over time.

Advantageously, the cardiac tissue according to the invention has a content of cells expressing cardiac troponin C of at least 50% by number relative to the total number of cells forming the compacted tissue, even more preferentially at least 60%, at least 70%, at least 75%, at least 80%, at least 90%. This high content of cells expressing cardiac troponin C is advantageous for envisaging uses of the cardiac tissues according to the invention in cell therapy.

Preferentially, the compacted tissue of human cardiac cells according to the invention has a content of cells expressing alpha-actinin of at least 50% by number relative to the total number of cells forming the compacted tissue, even more preferentially at least 60%, at least 70%, at least 75%, at least 80%, it being possible for this content to be greater than 90%. This high content of cells expressing alpha-actinin is advantageous for envisaging uses of the cardiac tissues according to the invention in cell therapy.

Preferentially, the compacted tissue of human cardiac cells according to the invention has a content of cells expressing troponin C and alpha-actinin of at least 50% by number relative to the total number of cells forming the compacted tissue, even more preferentially at least 60%, at least 70%, at least 75%, at least 80%, it being possible for this content to be greater than 90%. This high content of cells expressing both cardiac troponin C and alpha-actinin is advantageous for envisaging uses of the cardiac tissues according to the invention in cell therapy.

FIGS. 7, 8 and 9 show that, for a given starting cell population, tissues obtained within a microcompartment/capsule proceeding via a phase undergoing differentiation with the presence of at least one lumen, then a secondary compaction, have a much greater content of cells expressing troponin C compared to tissues differentiated using the same protocol (and the same initial batch of human pluripotent stem cells) but in free suspended culture. Thus, differentiation into cardiomyocytes in microcompartments and/or using a method comprising secondary compaction makes it possible to increase the quality of the cardiac tissues and therefore improves the possibility of using them in cell therapy.

The compacted cardiac tissue according to the invention can be obtained by any suitable method. According to one embodiment, it can be obtained from at least one cellular microcompartment of human cells undergoing cardiac differentiation, using a method comprising the compaction of the layer of human cells by the total or partial elimination of the lumen(s) of said microcompartment. Preferentially, the compacted tissue of human cardiac cells is obtained by a method as described previously.

The compacted tissue of human cardiac cells according to the invention can be entirely or partially encapsulated in an external hydrogel layer. The hydrogel capsule may be that from which the microcompartment of human cells undergoing cardiac differentiation originates, or it may be a new hydrogel layer if the initial hydrogel layer has been eliminated, then re-encapsulation has taken place at any stage of the method.

The encapsulation of the compacted tissue of human cardiac cells according to the invention makes it possible to protect the tissue, to maintain the spontaneous contraction frequency of less than 4 Hz, preferentially less than 2 Hz, even more preferentially less than 1 Hz, and in particular less than 0.5 Hz and which may even be less than 0.25 Hz. The mechanism by means of which the contraction frequency is limited may be related to the 3D structuring via i) creating electrical continuity of the cytoplasms of the cardiac cells, ii) and/or limiting the amount of calcium available per cell in the intercellular space of the compacted tissues, iii) and/or the mechanical strength related to the mechanical continuity of the cytoskeleton elements of the cardiac cells. The encapsulation of the compacted cardiac tissue according to the invention also makes it possible to control the size of the compacted tissue, which improves cell retention, integration and survival when it is injected into the heart, in particular compared to injections of single cells, thereby increasing the efficacy of the cardiac cell therapy with the compacted tissues according to the invention.

According to one embodiment, the compacted tissue of human cardiac cells according to the invention is not encapsulated in an external hydrogel layer. In particular, the capsule is preferentially eliminated before use in order to allow the cells of the compacted tissue to become implanted in the heart following a graft. Preferentially, the hydrogel used is biocompatible, that is to say it is non-toxic to the cells. According to one embodiment, the external hydrogel layer comprises at least alginate. It may consist exclusively of alginate. The alginate can in particular be a sodium alginate, composed of 80% α-L-guluronate and 20% β-D-mannuronate, with an average molecular weight of 100 to 400 kDa and a total concentration of between 0.5 and 5% by weight. The external layer is closed or partially closed.

The compacted tissue of human cardiac cells according to the invention is preferentially entirely or partially surrounded by a layer of isotonic aqueous solution. This layer of isotonic aqueous solution is located between the compacted tissue of human cardiac cells and the hydrogel layer when the compacted cardiac tissue is encapsulated. The layer of isotonic aqueous solution preferentially contains peptide or peptidomimetic sequences capable of binding to integrins. “Isotonic aqueous solution” means an aqueous solution having an osmolarity of between 200 and 400 mOsm/L. This isotonic aqueous solution preferentially contains peptide or peptidomimetic sequences capable of binding to integrins. It may in particular be an extracellular matrix or a culture medium. If it is an extracellular matrix, it may consist of extracellular matrix secreted by cells of the inner layer and/or of added extracellular matrix. The intermediate layer may form a gel. It preferentially comprises a mixture of proteins and extracellular compounds necessary for culturing cells undergoing cardiac differentiation. Preferentially, the aqueous solution comprises structural proteins, such as collagen, laminins, entactin, vitronectin, and growth factors, such as TGF-beta and/or EGF. According to one variant, the intermediate layer may consist of or comprise Matrigel® and/or Geltrex® and/or a hydrogel-type matrix of plant origin such as modified alginates or of synthetic origin or copolymer of poly(N-isopropylacrylamide) and poly(ethylene glycol) (PNIPAAm-PEG), of Mebiol® type.

At the surface of the compacted tissue in contact with the isotonic aqueous solution, the extracellular matrix may optionally contain one or more cells.

The thickness of the layer of isotonic aqueous solution around the compacted tissue according to the invention is preferentially between 30 nm and 300 μm, even more preferentially between 30 nm and 50 μm.

The compacted cardiac tissue according to the invention is three-dimensional. It preferentially has a spherical or elongated shape. According to a preferred embodiment, the compacted tissue of human cardiac cells has the shape of a spheroid, an ovoid, a cylinder or a sphere.

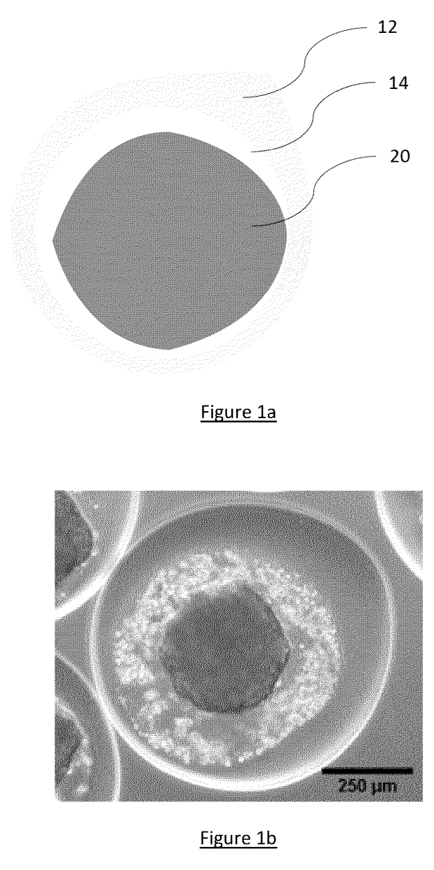

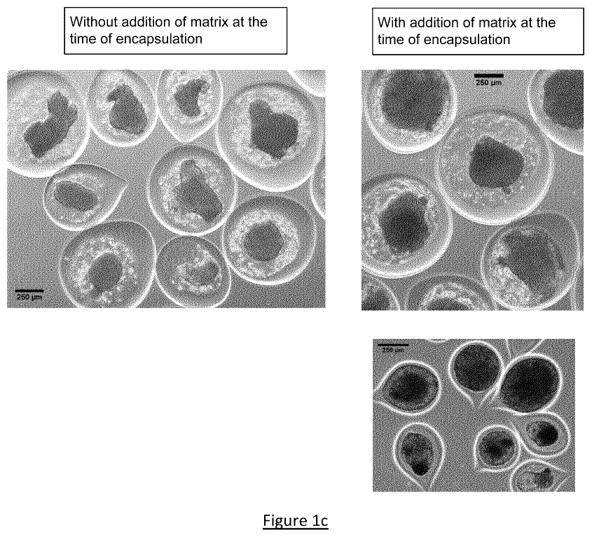

An example of compacted tissue according to the invention is shown in FIG. 1. In this example, the compacted tissue according to the invention is surrounded by a layer of isotonic aqueous solution and an external hydrogel layer.

It preferably has a diameter or a smallest dimension of between 10 μm and 1 mm, preferentially of between 100 μm and 700 μm. This smallest dimension is important for its survival, in particular to promote the survival of the cardiac cells within the cardiac tissue and to optimize reorganization as well as vascularization of the cardiac tissue after implantation in the heart.

Its largest dimension is preferentially greater than 10 μm, more preferentially between 10 μm and 1 m, even more preferentially between 10 μm and 50 cm. According to one embodiment, the largest dimension is compatible with the size of the organ and is therefore less than 30 cm (between μm and 30 cm).

The encapsulation of a controlled number of stem cells, and/or re-encapsulation, makes it possible to control the desired size and shape of the cardiac tissues obtained. Thus, the size of the cardiac tissues according to the invention can vary depending on the therapeutic use envisaged.

The compacted tissue of human cardiac cells according to the invention can be frozen to favor storage thereof.

Advantageously, the invention makes it possible to produce a large amount of quality human cardiac tissues by protecting the tissue units throughout their production by differentiation of pluripotent cells into cardiac cells.

The compacted tissue of cardiac cells according to the invention can be dissociated into cells. The dissociation can be carried out according to conventional methods known to those skilled in the art, in particular using an enzymatic solution that makes it possible to separate the cells. The enzymes used can, for example, be selected from trypsin, collagenase, accutase and mixtures thereof. The dissociated cells are preferentially used in suspension or integrated into a gel, for example a collagen gel in a patch.

### Method for Obtaining Compacted Cardiac Tissue

The cardiac tissue according to the invention can be obtained by any suitable method.

According to a particular embodiment, the compacted cardiac tissue according to the invention is obtained from a microcompartment of human cells undergoing cardiac differentiation.

This may in particular be a cellular microcompartment comprising in succession, organized around at least one lumen:
at least one inner layer of human cells undergoing cell differentiation into cardiac cells, expressing at least one gene selected from PDGFRα, MESP-1, NKX2-5, GATA4, MEF2C, TBX20, ISL1 and TBX5 (referred to as “inner layer”),
at least one intermediate layer of isotonic aqueous solution (referred to as “intermediate layer”), and
at least one external hydrogel layer (referred to as “external layer”).

The microcompartment is therefore a three-dimensional structure comprising at least one inner layer of cells. These cells are living human cells undergoing cell differentiation into cardiac cells. This layer of cells is organized in three dimensions in the microcompartment.

Human cells undergoing cardiac differentiation present in the microcompartment are cells expressing at least one gene selected from PDGFRα, MESP-1, NKX2-5, GATA4, MEF2C, TBX20, ISL1 and TBX5. These genes are specific to cardiac cells undergoing differentiation. Preferentially, the human cells undergoing cardiac differentiation present in the microcompartment express at least two of these genes. According to a variant, the human cells undergoing cardiac differentiation present in the microcompartment express all of these genes.

The inner layer of human cells undergoing cell differentiation into cardiac cells has a variable thickness. Within the microcompartment, the inner layer of human cells and the lumen(s) together form a three-dimensional cellular object. If the smallest and the greatest thickness of the inner layer of cells are measured along a segment passing through the geometric center of this cellular object, the ratio of the greatest thickness to the smallest thickness is greater than or equal to 2. The thicknesses of the inner layer are measured along the segment passing through the geometric center of the cellular object:
a. between:
the interface of the inner layer and the intermediate layer, and
the interface of the inner layer and a lumen,
   and/or
b. between:
the interface of the inner layer and a lumen, and
the interface of the inner layer and another lumen.

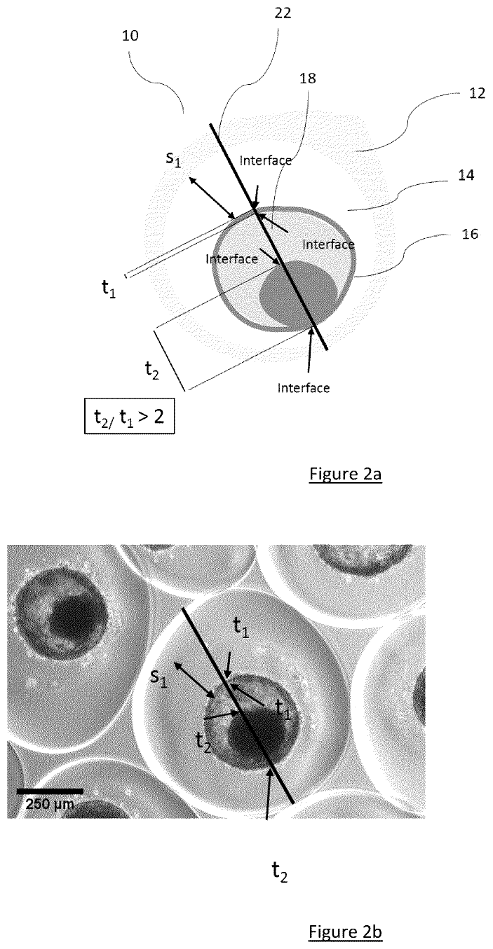

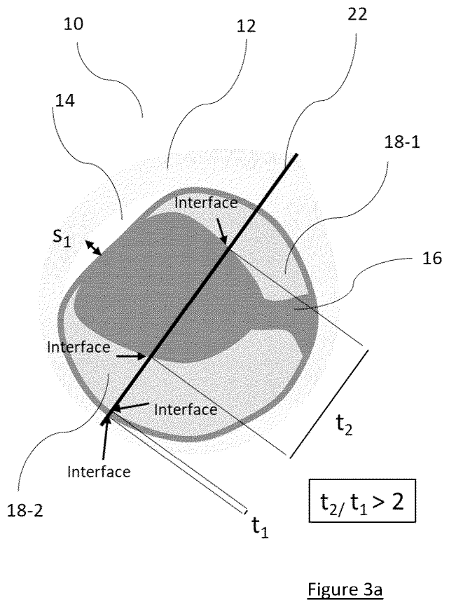

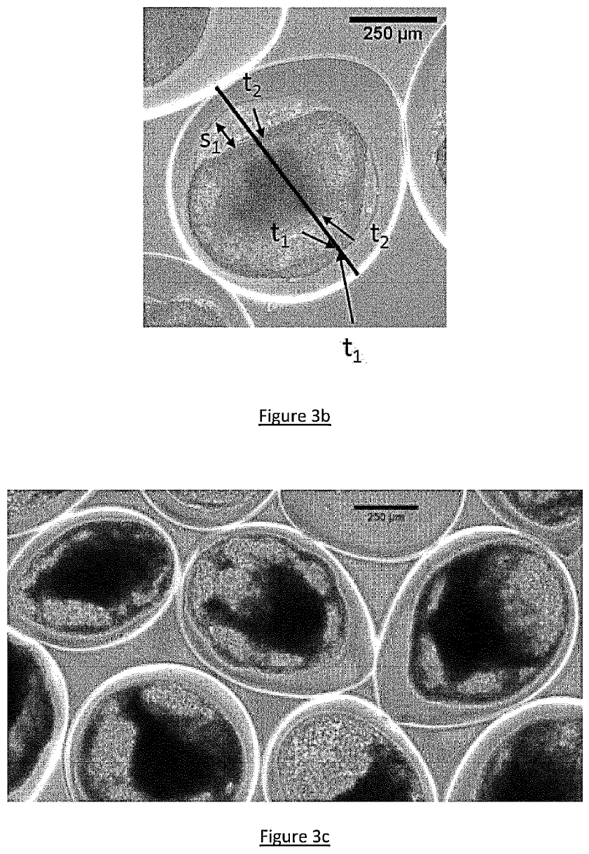

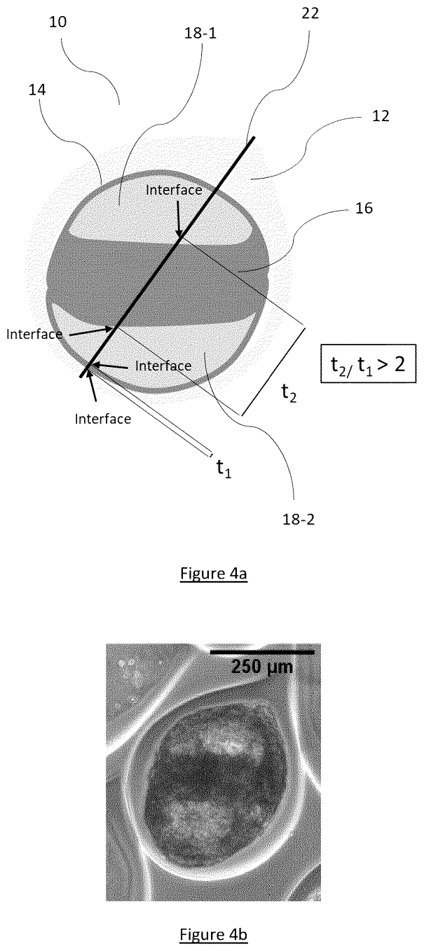

FIGS. 2, 3 and 4 show examples of such cellular microcompartments 10, with an external hydrogel layer 12, a layer of isotonic aqueous solution 14, one or more inner lumen(s) 18, 18-1, 18-2, a layer of human cells undergoing cardiac differentiation 16 with a greatest thickness t2 and a smallest thickness t1 (the thicknesses being measured along a segment 22 passing through the geometric center of this cellular object formed by the layer 16 and the lumen(s) 18, 18-1, 18-2), the t2/t1 ratio being much greater than 2.

In FIG. 2, there is only one lumen 18 and consequently the thicknesses of the inner layer are measured along a segment 22 passing through the geometric center of the cellular object formed by the layer 16 and the lumen 18, between the interface of the inner layer and the intermediate layer and the interface of the inner layer and the lumen 18.

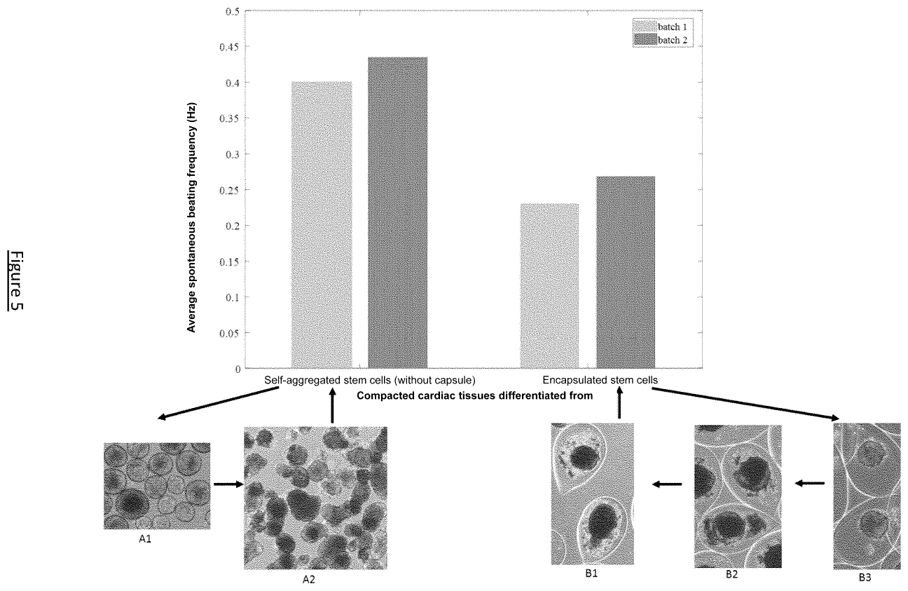

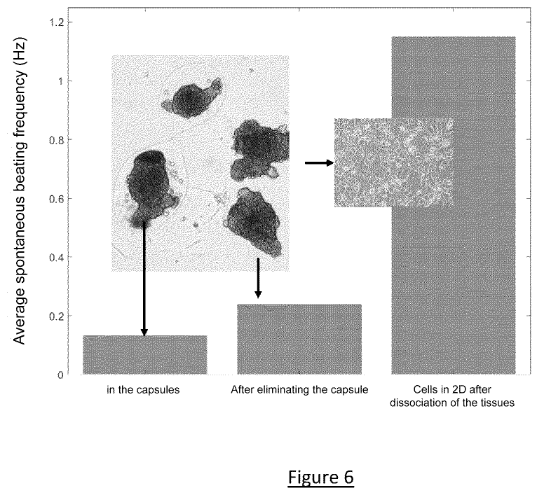

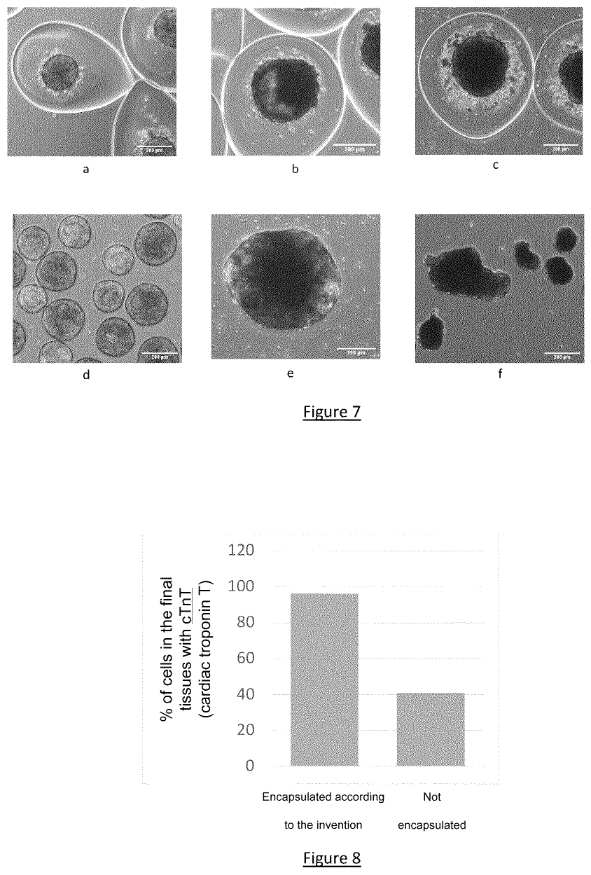

In FIGS. 3 and 4 there are two lumens 18-1 and 18-2 and consequently the thicknesses of the inner layer are measured along a segment 22 passing through the geometric center of the cellular object formed by the layer 16 and the lumens 181-18-2:
between the interface of the inner layer and the intermediate layer and the interface of the inner layer and the lumen 18-1, and
between the interface of the inner layer and the intermediate layer and the interface of the inner layer and the lumen 18-2, and
between the interface of the inner layer and the lumen 18-1 and the interface of the inner layer and the lumen 18-2.

The number of human cells undergoing cell differentiation into cardiac cells of the inner layer is preferentially between 1 and 100,000 cells, even more preferentially between 50 and 50,000 cells, and particularly between 500 and cells.

The human cells undergoing cell differentiation into cardiac cells of the inner layer were preferentially obtained from pluripotent stem cells, in particular human pluripotent stem cells, or optionally from non-pluripotent human cells, the transcriptional profile of which was artificially modified to match that of cardiac progenitors or cardiac cells, typically by forced expression of specific transcription factors for the target cellular phenotype. Preferentially, the human cells of the inner layer were obtained from human pluripotent stem cells after bring into contact with a solution capable of initiating the differentiation of said stem cells.

The intermediate layer of isotonic aqueous solution preferentially contains peptide or peptidomimetic sequences capable of binding to integrins. “Isotonic aqueous solution” means an aqueous solution having an osmolarity of between 200 and 400 mOsm/L. This layer is located between the inner layer of cells and the external hydrogel layer.

The intermediate layer may consist of elements which have been added during the production of the microcompartment and/or of elements added to the microcompartment and/or of elements secreted or induced by the other constituents of the microcompartment.

The intermediate layer may in particular comprise or consist of an extracellular matrix and/or a culture medium. If it comprises extracellular matrix, this may be extracellular matrix secreted by cells of the inner layer and/or by extracellular matrix added at the time of the preparation/production of the microcompartment.

The intermediate layer preferentially comprises a mixture of proteins and extracellular compounds necessary for culturing cells undergoing cardiac differentiation. Preferentially, the intermediate layer comprises structural proteins, such as collagen, laminins, entactin, vitronectin, and growth factors, such as TGF-beta and/or EGF. According to one variant, the intermediate layer may consist of or comprise Matrigel® and/or Geltrex® and/or a hydrogel-type matrix of plant origin such as modified alginates or of synthetic origin or copolymer of poly(N-isopropylacrylamide) and poly(ethylene glycol) (PNIPAAm-PEG), of Mebiol® type.

According to one variant, the intermediate layer may form a gel.

At the surface of the intermediate layer in contact with the inner layer of human cells undergoing differentiation, the extracellular matrix may optionally contain one or more cells.

The thickness of the intermediate layer in the microcompartments (shown as S1 in FIGS. 2 and 3) is preferentially between 30 nm and 300 μm, even more preferentially between 30 nm and 50 μm.

The presence of the intermediate layer promotes the structuring, according to the invention, of the elements in the microcompartment.

The microcompartment and the inner layer of cells undergoing cardiac differentiation within the microcompartment are hollow. Indeed, the microcompartment always comprises at least one inner lumen which constitutes the hollow part of the microcompartment. The lumen contains a liquid, in particular a culture medium (for example an RPMI basal medium with a B27 supplement) and/or a liquid secreted by the cells of the inner layer. Advantageously, the presence of this hollow part enables the cells to have a small diffusive volume of which they can control the composition, promoting what is referred to as autocrine/paracrine cell communication which is in turn favorable to cardiac differentiation.

According to one embodiment, as shown in FIGS. 3 and 4, the microcompartment may comprise a plurality of lumens, at least two lumens. This situation has the same advantage with respect to autocrine and paracrine signals as the presence of a single lumen and increases the capacity of the cells to control the composition of the aqueous solution of the lumen since the ratio of cells per volume/cells is then geometrically lower. Furthermore, the stabilization of such a configuration demonstrates the mechanical protection offered by the microcompartment.

The lumen(s) preferentially represent(s) between 10% and 90% of the volume of the microcompartment.

The microcompartment comprises an external hydrogel layer. Preferentially, the hydrogel used is biocompatible, that is to say it is non-toxic to the cells. The hydrogel layer must allow the diffusion of oxygen and nutrients in order to supply the cells contained in the microcompartment and to enable them to survive. According to one embodiment, the external hydrogel layer comprises at least alginate. It may consist exclusively of alginate. The alginate can in particular be a sodium alginate, composed of 80% α-L-guluronate and 20% β-D-mannuronate, with an average molecular weight of 100 to 400 kDa and a total concentration of between 0.5 and 5% by weight.

The hydrogel layer makes it possible to protect the cells from the external environment, to limit uncontrolled proliferation of the cells, and enables the controlled differentiation thereof into cells undergoing cardiac differentiation and then into cardiac cells, at least into cardiomyocytes.

The external layer is closed or partially closed. The microcompartment is therefore closed or partially closed. Preferentially, the microcompartment is closed.

The microcompartment may be in any three-dimensional form, that is to say it may have the shape of any object in space. Preferentially, the microcompartment is in a spherical or elongated shape. It may in particular be in the form of a hollow spheroid, a hollow ovoid, a hollow cylinder or a hollow sphere.

It is the external layer of the microcompartment, that is to say the hydrogel layer, which gives the microcompartment its size and shape. Preferentially, the diameter the smallest dimension of the microcompartment is between 10 μm and 1 mm, preferentially between 100 μm and 700 μm. It may be between 10 μm and 600 μm, in particular between 10 μm and 500 μm. This smallest dimension is important for the survival of the three-dimensional cardiac tissue which will be obtained from the microcompartment, in particular to promote the survival of the cardiac cells within the cardiac tissue and to optimize reorganization as well as vascularization of the cardiac tissue after implantation in the heart.

Its largest dimension is preferentially greater than 10 μm, more preferentially between 10 μm and 1 m, even more preferentially between 10 μm and 50 cm. According to one embodiment, the largest dimension is compatible with the size of the organ and is therefore less than 30 cm (between 10 μm and 30 cm).

The microcompartment is particularly useful for obtaining a three-dimensional compacted cardiac tissue, consisting of differentiated human cardiac cells.

The microcompartment may optionally be frozen for storage. It must then be thawed in order to continue the maturation of the cardiac cells and to obtain a compacted cardiac tissue according to the invention.

The microcompartments are preferentially used in series (at least two microcompartments) in a culture medium, in particular in an at least partially convective culture medium. According to a particularly suitable embodiment, the compacted cardiac tissues that are the subject of the invention are obtained from a series of cellular microcompartments as described previously in a closed chamber, such as a bioreactor, preferentially in a culture medium in a closed chamber, such as a bioreactor.

The microcompartments as described previously, and which may be used to obtain compacted cardiac tissues, can be obtained in particular by a method as described below. Such a method consists in producing cellular microcompartments comprising a hydrogel capsule surrounding:
stem cells or progenitor cells capable of differentiating into cardiac cells, at least into cardiomyocytes, or
differentiated cells intended to undergo, in the capsule, reprogramming in the capsule so that they become induced pluripotent stem cells capable of differentiating into cardiac cells, at least into cardiomyocytes.

According to one embodiment, the method for preparing a microcompartment can comprise at least the implementation of the steps consisting in:
producing a cellular microcompartment comprising, inside a hydrogel capsule:
elements of isotonic aqueous solution, preferentially of extracellular matrix, secreted by the cells or supplied by the operator, preferentially at least part of the isotonic aqueous solution being supplied in addition to the extracellular matrix naturally secreted by the cells,
cells capable of differentiating into cardiac cells,
inducing cell differentiation within the cellular microcompartment, so as to obtain at least one hollow three-dimensional layer of human cells undergoing cell differentiation into cardiac cells, expressing at least one gene selected from PDGFRα, MESP-1, NKX2-5, GATA4, MEF2C, TBX20, ISL1 and TBX5, and optionally other cells.

Advantageously, the total or partial encapsulation in the hydrogel combined with supplying extracellular matrix is a suitable means for enabling the differentiation of human pluripotent cells into cardiac muscle by accumulating several advantages, in particular:
i) promoting homogeneous distribution of the cells of the batch within the microcompartments,
ii) mechanical protection against hydrodynamic stress inflicted by the bioreactor and limiting undesired fusions of microcompartments,
iii) organization of a microenvironment that locally retains the extracellular matrix elements, promoting good survival and good cell organization,
iv) maintaining a lumen, promoting the autocrine and paracrine pathways during differentiation.

Use may be made of any method for producing cellular microcompartments containing, inside a hydrogel capsule, at least human cells undergoing cardiac differentiation and an isotonic aqueous solution and optionally adding other cells, for example support cells. A suitable method is described in particular in application WO 2018/096277. Preferentially, the encapsulation is carried out by co-injecting three solutions:
a hydrogel solution,
an intermediate isotonic solution, for example a sorbitol solution,
a solution comprising the cells to be encapsulated, the culture medium and optionally but preferentially the extracellular matrix,

concentrically via a microfluidic injector which makes it possible to form a jet at the outlet of the injector consisting of the mixture of the three solutions, said jet breaking up into droplets, said droplets being collected in a calcium bath which stiffens the hydrogel solution to form the external layer of each microcompartment, the inner part of each droplet consisting of the solution comprising the encapsulated cells, the culture medium and the extracellular matrix.

According to one embodiment, the encapsulation is carried out with a device capable of generating hydrogel capsules using a microfluidic chip. For example, the device may comprise syringe pumps for several solutions injected concentrically by virtue of a microfluidic injector, which makes it possible to form a jet which breaks up into droplets which are then collected in a calcium bath. According to a particularly suitable embodiment, three solutions are loaded on three syringe pumps:
a hydrogel solution, for example alginate,
an intermediate isotonic solution, for example a sorbitol solution,
the solution resulting from step b) comprising iPSCs, the culture medium and optionally but preferentially the extracellular matrix

The three solutions are co-injected (simultaneously injected) concentrically by virtue of a microfluidic injector or microfluidic chip which makes it possible to form a jet that breaks up into droplets, the external layer of which is the hydrogel solution and the core of the solution comprising the cells to be encapsulated; these droplets are collected in a calcium bath which stiffens the alginate solution to form the shell.

To improve the monodispersity of the cellular microcompartments, the hydrogel solution is charged with a direct current. A ring at ground is arranged after the tip in the plane perpendicular to the axis of the jet emerging from the microfluidic injector (coextrusion chip) in order to generate the electric field.

In a particular embodiment, the step of producing a cellular microcompartment comprises the steps consisting in:
incubating pluripotent stem cells in a culture medium, preferentially a culture medium containing the growth factors FGF2 and TGFβ or molecules reproducing the action thereof on the cell, an inhibitor of the Rho kinase pathway or a molecule reproducing the action thereof on the cell, in particular by limiting cell death.
optionally mixing the pluripotent stem cells with an isotonic aqueous solution, preferentially an extracellular matrix,
encapsulating the mixture in a hydrogel layer.

The cells encapsulated for the preparation of microcompartments are preferentially selected from:
cells capable of differentiating at least into cardiac cells, these cells being:
either stem cells capable of differentiating into cardiac cells, at least into cardiomyocytes, preferentially embryonic stem cells or induced pluripotent stem cells, very preferentially induced pluripotent stem cells, and/or
or progenitor cells capable of differentiating into cardiac cells, at least into cardiomyocytes,
and/or differentiated cells capable of undergoing reprogramming so that they become induced pluripotent stem cells capable of differentiating into cardiac cells, at least into cardiomyocytes.

The encapsulated cells may be immunocompatible with the person intended to receive the differentiated cardiac cells obtained from the microcompartment, to avoid any risk of rejection. In one embodiment, the encapsulated cells were previously taken from the person in whom the compacted cardiac tissues according to the invention will be implanted.

The differentiation into cells undergoing cardiac differentiation contained in the microcompartment can be carried out by any suitable method. It may in particular be a known method, such as one of the protocols listed in (Dunn, K K & Palecek, S P Engineering fabrication évolutive de cardiomyocytes dérivés de cellules souches de haute qualitépour la réparation des tissus cardiaques. [Evolutive production of cardiomyocytes derived from high-quality stem cells for the repair of cardiac tissues] Front. Med. 5, (2018)).

The protocol which is currently one of the most common is described in detail in (Burridge, P. W. et al. Génération chimiquement définie de cardiomyocytes humains. [Chemically defined generation of human cardiomyocytes] Nat. Methods 11,855-860 (2014)).

In a particular embodiment, the step of inducing cell differentiation of the method comprises a step consisting in introducing capsules containing human stem cells capable of being differentiated into human cardiac cells into a culture medium containing a Wnt pathway activator (such as CHIR99021) for 12 h to 72 h, more preferentially 12 to 48 hours.

Next, the method may comprise a step which consists in incubating the microcompartments in a culture medium containing a Wnt pathway inhibitor. This step is preferentially carried out between 0 and three days after the end of the step of inducing differentiation, preferentially between 12 and 72 hours, in particular between 24 and 48 h. According to a preferred embodiment, this step consists in incubating the capsules in a culture medium containing a Wnt pathway inhibitor, preferentially for 12 hours to 48 hours, in particular between 24 and 48 h.

One particular embodiment is as follows:
(a) incubating human pluripotent stem cells containing capsules in a culture medium containing a Wnt pathway activator for 12 h to 72 h;
(b) 0 to 48 hours after step (a), incubating the capsules in a culture medium containing a Wnt pathway inhibitor for 24 hours to 48 hours;

Preferentially, the culture medium is RPMI with a B27 supplement, without insulin (during the first 7 days of differentiation) and with insulin (from differentiation day 7).

Preferentially, the microcompartments containing cells undergoing cardiac differentiation are obtained between 2 to 7 days after the start of inducing differentiation, preferentially between 3 and 7 days after the start of inducing differentiation, even more preferentially between 4 and 6 days after the start of differentiation. Preferentially, the microcompartment according to the invention appears at the moment that the Wnt pathway inhibitor is added, or thereafter.

The lumen is created at the moment of formation of the layer of human cells undergoing cardiac differentiation in 3 dimensions, by the cells that are multiplying and growing. The lumen can contain a liquid and in particular the culture medium used for the implementation of the method.

According to one embodiment, the starting stem cells are organized into a layer of stem cells in three dimensions around a lumen in the microcompartment, then during differentiation this lumen disappears, and a second lumen appears to form the microcompartment.

The method is preferentially implemented in a closed chamber, such as a bioreactor, with a series of microcompartments, even more preferentially in a suitable culture medium that is at least partially convective.

The method may also optionally comprise:
a step which consists in dissociating the microcompartment or the series of microcompartments in order to obtain a suspension of cells or a suspension of cell clusters; the capsule can be eliminated in particular by hydrolysis, dissolution, piercing and/or breaking by any biocompatible means, that is to say means which are not toxic for the cells. For example, the elimination may be accomplished using phosphate-buffered saline, a divalent ion chelator, an enzyme such as alginate lyase if the hydrogel comprises alginate, and/or laser microdissection, and
a step of re-encapsulating all or part of the cells or cell clusters in a hydrogel capsule.

The re-encapsulation is a suitable means for:
i) optimizing the standardization of the size and homogeneity of the compacted cardiac tissues that will then be obtained,
ii) enabling an increase in the cell amplification obtained from the pluripotent step, and therefore a higher yield.

At any time, the method may comprise a step consisting in verifying the phenotype of the cells contained in the microcompartment. This verification can be carried out by identifying the expression, by at least some of the cells contained in the microcompartment, of at least one of the following genes: PDGFRα, MESP-1, NKX2-5, GATA4, MEF2C, TBX20, ISL1 and TBX5.

The method may comprise a step of freezing the microcompartments before they are used to continue differentiation into differentiated cardiac cells and to obtain compacted cardiac tissues according to the invention. The freezing is preferentially carried out at a temperature of between −190° C. and −80° C. The thawing can be carried out in a warm water bath (37 degrees preferentially) in order for the cells to thaw quite rapidly. The microcompartments according to the invention, before they are used to continue differentiation into differentiated cardiac cells and to obtain compacted cardiac tissues, can be kept at more than 4° C. for a limited period of time before they are used, preferentially between 4° C. and 38° C.

The microcompartments can also be used to continue differentiation into differentiated cardiac cells and to obtain compacted cardiac tissues according to the invention, directly after implementation of the method, without storage and without freezing.

According to one variant, the cardiac differentiation within the microcompartment is possibly implemented and/or combined with other techniques, such as electrical stimulation and metabolic or hormonal interventions. The combination with such techniques can make it possible to further reduce the frequency of the spontaneous beating of the compacted cardiac tissue according to the invention before grafting.

After obtaining a microcompartment containing human cells undergoing differentiation as described above, the method can be continued in order to obtain a three-dimensional object in the form of a compacted cardiac tissue according to the invention. The compacted cardiac tissue according to the invention can in particular be obtained from at least one cellular microcompartment of human cells undergoing cardiac differentiation as described previously, using a method comprising the compaction of the layer of human cells by the total or partial elimination of the lumen(s) of said microcompartment. Preferentially, the compacted tissue of human cardiac cells is obtained by a method as described below.

The compacted object generally appears between 2 and 10 days after the addition of the Wnt pathway inhibitor, in particular between 5 and 7 days. Indeed, the addition of the inhibitor is preliminary to the compaction of the cells which continue to differentiate into cardiac cells.

Thus, the compacted object generally appears between 7 and 14 days after initiation of differentiation.

At the end of the compaction, all or some of the lumens have partially or entirely disappeared (have been partially or totally eliminated by the compaction phenomenon) and the cells at least in part comprise differentiated human cardiac cells, preferentially at least cardiomyocytes.

The method may comprise a step of amplifying the cardiac cells in the microcompartment, and optionally one or more re-encapsulations.

The obtained compacted cardiac tissue according to the invention can be maintained in the hydrogel capsule. Preferentially, it is always surrounded by an isotonic aqueous solution, preferentially an extracellular matrix. A capsule containing a three-dimensional compacted cardiac tissue according to the invention and a layer of isotonic aqueous solution is shown in FIG. 1.

The compacted tissue according to the invention is preferentially stored in a capsule before use. For its storage, the capsule containing the compacted cardiac tissue according to the invention can be frozen before eliminating the hydrogel layer from the capsule. The method can thus comprise a step of freezing the capsules containing the compacted cardiac tissues according to the invention before use thereof. The freezing is preferentially carried out at a temperature of between −190° C. and −80° C. The capsules containing the compacted cardiac tissues according to the invention before use thereof as a graft in the heart, may be thawed in a warm water bath (37 degrees preferentially) in order for the cells of the tissue to thaw quite rapidly. The compacted cardiac tissues according to the invention can be kept at more than 4° C. for a limited period of time before they are used, preferentially between 4° C. and 38° C.

After obtaining the compacted cardiac tissue according to the invention, at any time prior to implantation in the heart, the method may comprise a step consisting in verifying the phenotype of the cells contained in the capsule. This verification can be carried out by identifying the expression of cardiac troponin C by the cardiac cells forming the compacted tissue according to the invention.

Preferentially, before use, the hydrogel layer of the capsule containing the compacted cardiac tissue according to the invention is eliminated. The capsule can be eliminated in particular by hydrolysis, dissolution, piercing and/or breaking by any biocompatible means, that is to say means which are not toxic for the cells. For example, the elimination may be accomplished using phosphate-buffered saline, a divalent ion chelator, an enzyme such as alginate lyase if the hydrogel comprises alginate, and/or laser microdissection.

The hydrogel is preferentially entirely eliminated; the compacted cardiac tissue according to the invention is devoid of hydrogel when it is used as a graft, implanted in a heart.

### Uses of the Compacted Tissue of Human Cardiac Cells According to the Invention

The compacted tissue of human cardiac cells according to the invention can be used as is or to produce a suspension of cardiac cells.

Indeed, the compacted tissue of human cardiac cells according to the invention is particularly useful for the production of a suspension of cells (graft cells) which can be implanted into a human heart, in particular for treating cardiac pathologies. The shape, size and composition of the compacted tissue according to the invention promote homogeneous differentiation with an improved yield of cardiac cells within the compacted tissue according to the invention, which may be secondarily dissociated before implantation in the heart.

The compacted tissue of human cardiac cells according to the invention is also particularly useful for the use thereof as a graft which can be implanted into a human heart, in particular for treating cardiac pathologies. The shape, size and composition of the compacted tissue according to the invention enable the survival of cardiac cells within the compacted tissue according to the invention before implantation, and the successful implantation, reorganization and vascularization of the graft once implanted in the heart.

Another subject of the invention is therefore the compacted tissue of human cardiac cells for use thereof, as is or after dissociation, in the form of a suspension of cells, in therapy, in particular in cell therapy, as a medicament, in particular the use thereof in the treatment and/or prevention of a cardiac pathology, particularly in a patient in need of same, and preferentially in the treatment and/or prevention of ischemic heart disease.

Although it is possible to use dissociated cells obtained from tissues according to the invention, they have a higher spontaneous contraction frequency than the compacted cardiac tissues. The slow spontaneous beating rate of the differentiated cardiomyocytes within the capsule is not maintained when the cells are dissociated and cultured under 2D conditions (FIG. 6).

Treatment means a preventive, curative or symptomatic treatment, that is to say any act intended to improve a person's sight temporarily or permanently, and preferentially also to eradicate the disease and/or stop or delay the progression of the disease and/or promote the regression of the disease.

Indeed, the compacted tissues of human cardiac cells according to the invention can be used for the treatment of heart diseases in humans, in particular diseases which have led to ischemia of at least part of the heart, such as infarction, for example, to replace damaged areas.

The treatment consists in implanting, grafting the compacted tissues according to the invention, or the cells obtained by dissociation thereof, into the heart, at the heart ventricles, in particular the left ventricle, or in integrating them into a patch positioned on said ventricles, ideally between the visceral pericardium and the muscle tissue of the ventricle, or what remains thereof in a disease state. Use is very preferentially made of a surgical implantation device suitable for implantation in the heart. This may in particular be needles, cannulas or other devices which make it possible to deposit the compacted tissues according to the invention, or the cells obtained by dissociation of the compacted tissues according to the invention, in the heart, for example those used for the implantation of stents in arteries or of surgical microimplants.

According to one exemplary embodiment, the implantation can be carried out by direct myocardial injection, in particular by sternotomy or with a catheter-based device: the compacted cardiac tissues according to the invention or the cells obtained from these tissues (with or without the addition of other types of cells) are injected into the patient's left ventricular medial wall at one or more locations.

According to another exemplary embodiment, the implantation can be carried out using an epicardial patch. The compacted cardiac tissues according to the invention or the cells obtained from these tissues (with or without the addition of other types of cells) are used in the formation of patches.

These patches can then be placed on the epicardial surface of the patient's left ventricle, either by sternotomy or by a surgical procedure involving incision and injection of the patch into the thoracic cavity.

In one embodiment, during the same implantation, between 1 and 1,000,000 compacted tissue units according to the invention are implanted.

In one embodiment, during the same implantation, between 10⁴ and 10¹⁰ cells obtained by dissociation of compacted tissue units according to the invention are implanted.

If necessary, it is possible to perform a plurality of simultaneous or successive implantations in different areas of the heart, in particular in the event that several separate areas are affected or if the area to which the graft is to be applied is too extensive to perform grafting at only one location.

Likewise, in the same area, if a single graft is insufficient, several implantations can again be performed in the same area, over a longer or shorter period of time.

The implantation of compacted cardiac tissues according to the invention makes it possible for patients suffering from heart diseases, and in particular ischemic heart diseases, to clinically improve heart function, in particular:
to increase the contraction performance of the heart cells (which may be measured for example by the left ventricle ejection fraction) and/or
to increase the thickness of the ventricular wall.

Thus, advantageously, the invention makes it possible to improve the patient's overall health and quality of life, while limiting the risk of arrhythmias induced by the graft.

According to another aspect, the compacted tissues according to the invention can be useful as a cardiac tissue model, in particular:
to test medicaments and medicament candidates for efficacy in cardiac pathologies and/or the effect thereof on the heart, and/or
to test the cardiac toxicity of substances, compounds, compositions or medicaments. Thus, another subject of the invention is these uses.

The invention will now be illustrated using examples.

### EXAMPLES

Several examples of compacted tissues according to the invention are presented in FIGS. 1ato 1c, and examples of microcompartments capable of being used to obtain said compacted tissues are presented in FIGS. 2 to 4.

### Example 1

The images of FIGS. 2b and 2c are phase contrast microscopy images of a microcompartment taken at 4× magnification. They were taken 5 days after the start of differentiation (8 days after the initial encapsulation of the stem cells). The steps used to obtain the microcompartment shown in these figures are as follows:
1. The human induced pluripotent stem cells were encapsulated in an alginate hydrogel (without adding extracellular matrix at the time of encapsulation).
2. The encapsulated stem cells were cultured in stem cell culture media (mTeSR1) for 3 days.
3. On the 3rd day, the culture medium was changed from the stem cell medium to a cardiac differentiation medium containing a Wnt-activating molecule (CHIR99021). The medium is an RPMI medium, supplemented with B27, without insulin, with CH IR99021. This is considered to be differentiation day 0.
4. On the 2nd day of differentiation, the medium was changed for a cardiac differentiation medium without a Wnt-activating molecule. The medium is an RPMI medium, supplemented with B27, without insulin.
5. On the 3rd day of differentiation, the medium was changed for a cardiac differentiation medium containing a Wnt pathway-inhibiting molecule (C59 or IWR1). The medium is an RPMI medium, supplemented with B27, without insulin, with Wnt-059 or IW R1.
6. On the 5th day of differentiation, the photos in FIGS. 2b and 2c were taken by phase contrast microscopy of a microcompartment taken at 4× magnification.

### Example 2

The images of FIGS. 3b and 3c are phase contrast microscopy images of a microcompartment taken at 4× magnification. They were taken 5 days after the start of differentiation (11 days after the initial encapsulation of the stem cells). The steps used to obtain the microcompartment shown in these figures are as follows:
1. The human induced pluripotent stem cells were encapsulated in an alginate hydrogel (with addition of extracellular matrix at the time of encapsulation).
2. The encapsulated stem cells were cultured in stem cell culture media (mTeSR1) for 6 days.
3. On the 6th day, the culture medium was changed from the stem cell medium to a cardiac differentiation medium containing a Wnt-activating molecule (CHIR99021). The medium is an RPMI medium, supplemented with B27, without insulin, with CHIR99021. This is considered to be differentiation day 0.
4. On the 2nd day of differentiation, the medium was changed for a cardiac differentiation medium without a Wnt-activating molecule. The medium is an RPMI medium, supplemented with B27, without insulin.
5. On the 3rd day of differentiation, the medium was changed for a cardiac differentiation medium containing a Wnt pathway-inhibiting molecule (C59 or IWR1). The medium is an RPMI medium, supplemented with B27, without insulin, with Wnt-059 or IWR1.
6. On the 5th day of differentiation, the photos in FIGS. 3b and 3c were taken by phase contrast microscopy of a microcompartment according to the invention taken at 4× magnification.

### Example 3

The image of FIG. 4a is a phase contrast microscopy image of a microcompartment taken at 4× magnification. It was taken 5 days after the start of differentiation (11 days after the initial encapsulation of the stem cells). The steps used to obtain the microcompartment shown in these figures are the same as cells to obtain FIGS. 3b and 3c. The difference lies in a larger number of encapsulated stem cells.

### Example 4

FIGS. 1b and 1c are phase contrast microscopy images taken at 4× magnification of compacted tissues according to the invention in hydrogel capsules. The compacted tissues according to the invention were obtained by continuing the differentiation beyond day 5 (already described for the previous figures):
1. On the 7th day of differentiation, the culture medium was replaced with an RPMI medium, supplemented with B27, with insulin.
2. The medium was changed every 2-3 days until imaging.

The images presented in FIG. 1 relate to compacted tissues 14 days after the start of the differentiation.

### Comparative Tests: Spontaneous Beating Rate

FIG. 5 shows that, for a given starting cell population, cardiomyocytes differentiated within the capsule (from encapsulated hiPSCs) have a spontaneous beating rate that is slower than cardiomyocytes differentiated using the same protocol (and from the same initial batch of hiPSCs) but in free suspended culture. The beating frequency (defined as 1 time between spontaneous beats) was obtained from a series of phase contrast microscopy images (at a frequency of at least 30 images per second) on a standard table microscope with 4× magnification. The images are phase contrast microscopy images taken at 4× magnification showing (the outermost) encapsulated or free stem cells at the start of differentiation, and the final compacted tissues approximately 2 weeks after the start of differentiation (the innermost). In the case of the encapsulated differentiation process, an intermediate step with a microcompartment according to the invention is presented on differentiation day 5.

FIG. 6 shows that the slow rate of spontaneous beating of the cardiomyocytes differentiated in the capsule is slightly increased after removing the capsule, and greatly increased after the cells have been dissociated and placed in 2D culture. Thus, the compacted cardiac tissues have a slower beating frequency than that of the isolated cells obtained by dissociation of said tissues. The beating frequency (defined as 1 time between spontaneous beats) was obtained from a series of phase contrast microscopy images (at a frequency of at least 30 images per second) on a standard table microscope with 4× magnification. The beating frequency for the compacted cardiac tissues according to the invention, encapsulated and then with the capsules removed, was taken approximately 3 weeks after the start of differentiation. Approximately 3 weeks after differentiation, a sub-population of compacted tissues according to the invention was dissociated and placed under 2D culture conditions for 1 week before recording the beating frequency. The images are phase contrast microscopy images taken at 4× magnification. The left-hand image shows the compacted cardiac tissues differentiated in the capsule from encapsulated hiPSCs. The right-hand image shows the cells obtained from a sub-set of the initial encapsulated compacted cardiac tissues after having been placed under 2D culture conditions.

### Comparative Tests: Topology, Cell Amplification and Number of Cells Expressing Troponin C

Comparative tests with the same differentiation method under the same experimental conditions were carried out to compare the differentiation into cardiac cells and the three-dimensional tissues obtained with or without encapsulation and with or without secondary compaction.

All the cultures were produced from the same initial cells taken off from a 2D culture. Aggregates obtained in 3D are cultured (without addition of matrix) with or without capsules in a stirred suspension culture (with stirring at 55 rpm). The same cell density is used at the start (e6 cells/mL of medium) on day 0 of the differentiation (images a) and e)). The same differentiation protocol is applied under both conditions (with and without capsule).

FIG. 7 presents phase contrast microscopy images taken at 4× magnification. The three images on the top row (a, b and c) are images of encapsulated cells according to the invention.

The three images on the bottom row (d, e and f) are images of non-encapsulated cells.

The images in the left-hand column (a and d) show stem cells induced at the start of differentiation into cardiac cells.

The images in the middle column (b and e) show human cells undergoing cell differentiation into cardiac cells 3 to 7 days after initiation of differentiation.

The images in the right-hand column (c and f) show differentiated cardiac tissues.

It is observed that the topology is different with and without encapsulation according to the invention. Without encapsulation, there is no lumen undergoing differentiation, and therefore no subsequent compaction, and the cardiac tissue obtained at the end of differentiation has a very different shape.

In FIG. 8, it is observed that the percentage of cells in the tissues (obtained as in FIGS. 7c and 7f) expressing cardiac troponin C is greater than 90% under the conditions of the invention (secondary compaction), whereas it is 40% for the cardiac tissues obtained under the same conditions but without encapsulation.

In FIG. 9, it is observed that the level of cell amplification between the start of the differentiation is greater than 2 under the conditions of the invention, whereas it is less than 0.5 for the cardiac tissues obtained under the same conditions but without encapsulation.

1. A three-dimensional compacted tissue of human cardia cells expressing cardiac troponin C, characterized in that it is contractile and has a spontaneous contraction frequency of less than 4 Hz.

2. The compacted tissue of human cardiac cells according to claim 1, characterized in that it has a content of cells expressing cardiac troponin C of greater than 50% by number relative to the total number of cells of the tissue.

3. The compacted tissue of human cardiac cells of claim 1, characterized in that it has a content of cells expressing cardiac troponin C and alpha-actinin of greater than 50% by number relative to the total number of cells of the tissue.

4. The compacted tissue of human cardiac cells of claim 1, characterized in that it has a content of cells expressing cardiac troponin C of greater than 75% by number relative to the total number of cells of the tissue.

5. The compacted tissue of human cardiac cells of claim 1, characterized in that it has a spontaneous contraction frequency of less than 0.5 Hz.

6. The compacted tissue of human cardiac cells of claim 1, characterized in that it has a spontaneous contraction frequency of less than 0.25 Hz.

7. The compacted tissue of human cardiac cells of claim 1, characterized in that it is at least partially surrounded by a layer of isotonic aqueous solution.

8. The compacted tissue of human cardiac cells of claim 1, characterized in that it is entirely or partially encapsulated in an external hydrogel layer.

9. The compacted tissue of human cardiac cells of claim 1, characterized in that it has a spherical or elongated shape.

10. The compacted tissue of human cardiac cells of claim 9, characterized in that it is a spheroid, an ovoid, a cylinder or a sphere.

11. The compacted tissue of human cardiac cells of claim 1, characterized in that it has a diameter or a smallest dimension of between 10 μm and 1 mm/

12. The compacted tissue of human cardiac cells of claim 1, characterized in that it has a largest dimension of between 10 μm and 50 cm.

13. The compacted tissue of human cardiac cells of claim 1, from a tissue of cardiac cells and/or cells undergoing cardiac differentiation comprising at least one lumen, by a method comprising the compaction of said tissue by the total or partial elimination of said lumens.

14. The compacted tissue of human cardiac cells of claim 1, characterized in that it is frozen.

15. The compacted tissue of human cardiac cells of claim 1, for use thereof, as is or after dissociation, in the form of a suspension of cells, in the treatment and/or prevention of a cardiac pathology.

16. The compacted tissue of human cardiac cells for use thereof according to claim 15 in the treatment and/or prevention of ischemic heart disease.

17. A use of a compacted tissue of human cardiac cells of claim 1:
to test medicaments and medicament candidates for efficacy in cardiac pathologies and/or the effect thereof on the heart, and/or
to test the cardiac toxicity of substances, compounds, compositions or medicaments.
